(19) United States
(12) Patent Application Publication
(10) Pub. No.: US 2026/0308520 A1
(43) Pub. Date: Oct. 8, 2026

(54) Small Molecule Based Method Of Modifying T Cells

(71) Applicant: AGENCY FOR SCIENCE, TECHNOLOGY AND RESEARCH, Singapore (SG)

(72) Inventors: Qiang Yu, Singapore (SG); Shijun Ma, Singapore (SG); Li Teng Ong, Singapore (SG)

(21) Appl. No.: 19/490,282

(22) PCT Filed: Jun. 7, 2024
(86) PCT No.: PCT/SG2024/050382

§ 371 Date: Dec. 4, 2025

(30) Foreign Application Priority Data
Jun. 7, 2023 (SG) 10202301612V

(51) Int. Cl. C12N 5/0783; A61K 40/11; A61K 40/31; A61K 40/32; A61K 40/42; A61P 35/00; C07K 16/28; C07K 16/32

(52) U.S. Cl. CPC C12N 5/0636; A61K 40/11; A61K 40/31; A61K 40/32; A61K 40/4204; A61K 40/4205; A61K 40/421; A61P 35/00; C07K 16/2803; C07K 16/2863; C07K 16/32; C12N 2501/71; C12N 2510/00

(57) ABSTRACT

This technology relates to a method of T cell modification for immunotherapy.

### CROSS-REFERENCE TO RELATED APPLICATIONS

This application claims priority to 10202301612V, filed on 7 Jun. 2023, which is incorporated herein by reference in its entirety.

### FIELD OF THE INVENTION

The present disclosure relates generally to the field of cell biology. In particular, the present disclosure relates to T cell reprogramming.

### BACKGROUND

Cellular immunotherapies for the treatment of cancers are under rapid development. For example, chimeric-antigen-receptor T cell (CAR-T) therapies have been approved by FDA for haematological cancers, which are now available to the patients in need. However, due to technological bottlenecks that limit the efficacy of CAR-T therapy in solid tumor and reduce productivity of CAR-T cells, no FDA-approved CAR-T therapy for solid tumor is available to date. Such technological limitations for conventional methods include the long manufacture duration to obtain functional CAR-T cells, which result in differentiation of T cells, reduced T cell stemness and reduced memory T cell population during ex vivo expansion. In addition, weak immune activation of T cells and T cell exhaustion in the adverse solid tumor microenvironment further limits the efficiency of CAR-T therapies in solid tumors. Therefore, what is needed is a method which improves T cell expansion and enhances T cell stemness and memory T cell enrichment for the purpose of immunotherapy. Furthermore, other desirable features and characteristics will become apparent from the subsequent detailed description and the appended claims, taken in conjunction with the accompanying drawings and this background of the disclosure.

### SUMMARY

In one aspect, the present disclosure provides a method of modifying T cells during T cell activation, comprising modifying T cells by contacting the T cells with an inhibitory agent for prolyl hydroxylase.

In another aspect, the present disclosure provides a method of preparing modified T cells, comprising modifying T cells during T cell activation by contacting the T cells with an inhibitory agent for prolyl hydroxylase, wherein the modifying comprises increasing the expansion rate of T cells compared to unmodified T cells.

In another aspect, the present disclosure provides a method of producing Chimeric Antigen Receptor (CAR) T cells, comprising: modifying T cells during T cell activation by contacting the T cells with an inhibitory agent for prolyl hydroxylase; introducing a CAR transgene into the modified T cells of (a) to produce CAR-T cells; and harvesting the CAR-T cells after (b).

In another aspect, the present disclosure provides a method of producing T Cell Receptor (TCR) T cells comprising: (i) modifying T cells during T cell activation by contacting the T cells with an inhibitory agent of prolyl hydroxylase; (ii) introducing a TCR transgene into the modified T cells of (i) to produce TCR-T cells; and (iii) harvesting the TCR-T cells after (ii).

In another aspect, the present disclosure provides a modified T cell obtained by the method as disclosed herein.

In another aspect, the present disclosure provides a CAR-T cell obtained by the method as disclosed herein.

In another aspect, the present disclosure provides a TCR-T cell obtained by the method as disclosed herein.

In another aspect, the present disclosure provides a method of treating or alleviating a cancer in a subject, comprising administering a therapeutically effective amount of a CAR-T cell as disclosed herein, or a TCR-T cell as disclosed herein to the subject in need thereof.

In another aspect, the present disclosure provides a method of inducing long-term anti-tumor effect in a subject having a cancer, comprising administering a therapeutically effective amount of a CAR-T cell as disclosed herein, or a TCR-T cell as disclosed herein to the subject in need thereof.

In another aspect, the present disclosure provides a method of reducing tumor size in a subject having a cancer, comprising administering a therapeutically effective amount of a CAR-T cell as disclosed herein, or a TCR-T cell as disclosed herein to the subject in need thereof.

In another aspect, the present disclosure provides a method of increasing memory T cell and CAR-T cell stemness in vivo in a subject, comprising administering a therapeutically effective amount of a CAR-T cell as disclosed herein, or a TCR-T cell as disclosed herein to the subject in need thereof.

In another aspect, the present disclosure provides a method of increasing tumor infiltration of T cells in a subject having a cancer, comprising administering a therapeutically effective amount of a CAR-T cell as disclosed herein, or a TCR-T cell as disclosed herein to the subject in need thereof.

In another aspect, the present disclosure provides a method of increasing the in vivo expansion rate of CAR-T cells or TCR-T cells in a subject, comprising administering a therapeutically effective amount of a CAR-T cell as disclosed herein, or a TCR-T cell as disclosed herein to the subject in need thereof.

### BRIEF DESCRIPTION OF THE DRAWINGS

FIG. 1 compares the cells generated by conventional CAR-T method, and the STEM-T method as described herein upon ex vivo expansion. The T-cells used for generating CAR-T or STEM CAR-T are obtained from peripheral blood mononuclear cells (PBMC) from anonymous donor subjects. FIG. 1A shows the T cell growth curve of both CAR-T and STEM CAR-T cells over 42 days. Comparing to CAR-T cells obtained from a conventional method, STEM CAR-T cells expand faster and result in higher total number of CAR-T cells.

FIG. 1B and FIG. 1C show the flow analysis results of cytokines interferon gamma (IFN-γ) and interleukin 2 (IL-2) expression, respectively in CAR-T and STEM CAR-T cells over 20 days. STEM CAR-T cells exhibit higher or comparable level of cytokine production, which mediates the immune response in the body. FIG. 1D provides a flow analysis of T cell factor 1 (TCF1) among the total number CAR-T cells. TCF1 marks the population of stem-like precursors CD8+ T cells characterized by high self-renewal capacity, proliferative potential, and polyfunctionality. Compared to conventional CAR-T, STEM CAR-T cells show higher stemness. FIG. 1E and FIG. 1F further provide the percentages of the stem cell-like memory T cell (T_{scm}) subtype and central memory T cell (T_{cm}) subtype, respectively, among total number of CAR-T cells. Therefore, the STEM CAR-T cells obtained using the STEM modification method as described herein exhibits advantageous characteristics compared to conventional CAR-T cells including: faster in vitro expansion, higher cytokine production, improved stemness, and increased memory T cell population.

FIG. 2 compares the effects of STEM CAR-T cells and conventional CAR-T cells after 6 rounds of co-culturing with target tumor cells. The T-cells used for generating CAR-T or STEM CAR-T are obtained from peripheral blood mononuclear cells (PBMC) from anonymous donor subjects. FIG. 2A shows the expression of exhaustion markers PD1 and Tim3 for both ROR1 targeting conventional CAR-T and STEM CAR-T cells after the 6^{th} co-culture with MDA-MB-231 cells. T cell exhaustion refers to a condition in which T cells lose their cell effector functions and self-renewal capacity, such as killing cancer cells or cells infected with a virus over prolonged activation. Exhausted T cells in cancer show high expression levels of inhibitory receptors, such as PD-1, CTLA-4, TIM-3, LAG-3, BTLA and TIGIT, and reduced effector cytokine production, such as IL-2, TNF-α, IFN-γ and GzmB. As demonstrated in FIG. 2A, a decrease for PD1 and Tim3 markers in STEM CAR-T cells shows reduced exhaustion in STEM CAR-T cells, thereby allowing prolonged actions of the STEM CAR-T cells in the subject. FIG. 2B measures the CAR-T mediated cytotoxicity for both ROR1 targeting conventional CAR-T and STEM CAR-T cells when co-cultured with MDA-MB-231 cells stably expressing luciferase. As shown in FIG. 2B, STEM CAR-T has higher cytotoxicity than conventional CAR-T cells as measured by luciferase-based cell cytotoxicity assay. The CAR-T cells are injected to NSG mice pre-injected with MDA-MB-231-LN cells which bear the target tumor xenograft. FIG. 2C shows the number of CAR-T or STEM CAR-T cells in mouse blood after 1, 2, 3, and 4 weeks after injection. The number of STEM CAR-T cells are higher compared to conventional CAR-T throughout the entire 28 days (4 weeks) after injection, indicating long lasting in vivo persistency. FIG. 2D shows tumor growth curves in mice with injection of conventional CAR-T and STEM CAR-T, respectively. Control mice are treated with vehicle, which is phosphate buffered saline (PBS). Among all groups, it is clear that STEM CAR-T group shows most tumor volume reduction within 19 days after infusion, comparing to conventional CAR-T method. Therefore, STEM CAR-T cells show reduced T cell exhaustion and cytotoxicity in vivo. Additionally, STEM CAR-T cells, after infusion in mice, show significant higher number of cell counts in blood (FIG. 2C), and results in stronger anti-tumor efficacy in a mouse model.

FIG. 3 compares exemplary CAR-T cells (HER2) produced using conventional CAR-T and STEM CAR-T methods as described herein for T cell exhaustion. The T-cells used for generating CAR-T or STEM CAR-T are obtained from peripheral blood mononuclear cells (PBMC) from anonymous donor subjects. Exhausted T cells in cancer show high expression levels of inhibitory receptors, such as PD1 and Tim3 which are used as markers for T cell exhaustion. The HER2 specific STEM CAR-T cells show reduction in the exhaustion markers, indicating delayed exhaustion compared to conventional CAR-T cells.

FIG. 4 investigates the cell proliferation rate of human epidermal growth factor receptor 2 (HER2)-specific STEM CAR-T cells upon treatment with prolyl hydroxylase inhibitors as exemplified herein. The T-cells used for generating CAR-T or STEM CAR-T are obtained from peripheral blood mononuclear cells (PBMC) from anonymous donor subjects. The manufacture of STEM CAR-T cells comprises the step of T cell activation, engineered receptor induction, and small molecule treatment with prolyl hydroxylase inhibitor. FIG. 4A shows the cell count after treatment of T cells with exemplary prolyl hydroxylase inhibitors 1,4-DPCA, 1,4-DPCA ethyl ester, Molidustat, Enarodustat, Roxadustat, IOX2 and Daprodustat. The STEM CAR-T cells show a higher number of cells as illustrated in CAR-T (HER2) cell count 12 days after treatment with prolyl hydroxylase inhibitors compared to DMSO-treated negative control (i.e. conventional CAR-T cells). FIG. 4B shows the cell count after treatment of T cells with exemplary prolyl hydroxylase inhibitors and concurrent CD3/28 antibody stimulation compared to a DMSO negative control (i.e. conventional CAR-T cells). The increased cell counts after treatment with exemplary prolyl hydroxylase inhibitors demonstrated increased proliferation/expansion rate of the STEM CAR-T cells, with T cell activation induced by the incubation with CD3/28 antibody. Therefore, the STEM method as described herein is capable of producing higher number of STEM CAR-T cells for the purpose of subsequent administration to the patient.

FIG. 5 measures the percentage cell population of each T cell subtype among total number of CAR-T cells in HER2-targeting STEM CAR-T 8 days after exemplary prolyl hydroxylase inhibitors treatment and CD3/28 antibody stimulation. The T-cells used for generating CAR-T or STEM CAR-T are obtained from peripheral blood mononuclear cells (PBMC) from anonymous donor subjects. STEM CAR-T cells treated with exemplary prolyl hydroxylase inhibitors show higher number of T cell factor 1 (TCF1) positive cells and naïve T cell (Tₙ). The number of stem central memory T cell (T_{scm}) and central memory T cell (T_{cm}) also increased among the total CAR-T cell population. The population percentage of effector T cell (T_{eff}) and effector memory T cell (Tₑₘ) are maintained at low percentage relative to the total CAR-T cell population, comparing to the DMSO treated negative control (conventional CAR-T cells). Naïve T (Tₙ) cells, stem cell-like memory T (T_{scm}) cells and central memory-like (T_{cm}) cells exhibit high levels of stemness, with exceptional capabilities of self-renewal, longevity, and multipotent differentiation, compared to highly differentiated and short-lived T effector memory-like (Tₑₘ) cells, and highly differentiated T effector-like (T_{eff}) cells. Thus, the STEM CAR-T cells as described herein show improved stemness compared to conventional CAR-T cells, as demonstrated by higher population percentage of less differentiated cell types (Tₙ, T_{cm}, T_{scm}) and lower population percentage of differentiated cell types (Tₑₘ, T_{eff}). The high proliferative and self-renewal capacity of the less differentiated cell types allows persisting proliferation in vivo after administration, leading to improved clinical outcome.

FIG. 6 shows that prolyl hydroxylase inhibitors increase proliferation and stemness in exemplary ROR1 targeting STEM CAR-T cells (while maintaining differentiated T cells (Tₑₘ and T_{eff}) at low percentage. The T-cells used for generating CAR-T or STEM CAR-T are obtained from peripheral blood mononuclear cells (PBMC) from anonymous donor subjects. The prolyl hydroxylase inhibitor used for generating STEM CAR-T in this figure is 1,4-DPCA-ethyl-ester. As shown in FIG. 6A, exemplary STEM CAR-T cells targeting receptor tyrosine kinase-like orphan receptor 1 (ROR1) proliferate faster compared to conventional CAR-T cells.

FIG. 6B, FIG. 6C, and FIG. 6D show an increase in the naïve T cell population (Tₙ), stem cell-like memory T (T_{scm}), and T central memory-like (T_{cm}) cells in STEM CAR-T (ROR1) cells compared to conventional CAR-T cells, indicating an increase in less differentiated cell subtypes. FIG. 6E and FIG. 6F show comparable low percentages of cell population of differentiated cell types (Tₑₘ, and T_{eff}, respectively) in STEM CAR-T (ROR1) cells compared to conventional CAR-T cells. A higher number of T cell factor 1 (TCF1) positive cells is observed in FIG. 6G for STEM CAR-T (ROR1) cells. Therefore, CAR-T cells produced by STEM method show increased proliferation and stemness compared to the conventional method.

FIG. 7 shows that prolyl hydroxylase inhibitors increase proliferation and stemness in exemplary HER2 targeting STEM CAR-T cells (while maintaining differentiated T cells (Tₑₘ and T_{eff}) at low percentage. The T-cells used for generating CAR-T or STEM CAR-T are obtained from peripheral blood mononuclear cells (PBMC) from anonymous donor subjects. The prolyl hydroxylase inhibitor used for generating STEM CAR-T in this figure is 1,4-DPCA-ethyl-ester. As shown in FIG. 7A, exemplary STEM CAR-T cells targeting HER2 proliferate faster compared to conventional CAR-T cells. FIG. 7B and FIG. 7C show an increase in the naïve T cell population (Tₙ) and stem cell-like memory T (T_{scm}) in STEM CAR-T (HER2) cells compared to conventional CAR-T cells, indicating an increase in less differentiated cell subtypes. FIG. 7D, FIG. 7E, and FIG. 8F show maintenance of the percentage of cell population of differentiated cell types (T_{cm}, Tₑₘ, and T_{eff}, respectively) at low levels in STEM CAR-T (HER2) cells compared to conventional CAR-T cells. A higher number of T cell factor 1 (TCF1) positive cells is observed in FIG. 7G for STEM CAR-T (HER2) cells. Therefore, CAR-T cells produced by STEM method show increased proliferation and stemness compared to the conventional method.

FIG. 8 shows increased pro-inflammatory cytokines secretion and lysis against antigen expressing cancer cells and delayed exhaustion status in ROR1 targeting STEM CAR-T cells compared to conventional CAR-T. The T-cells used for generating CAR-T or STEM CAR-T are obtained from peripheral blood mononuclear cells (PBMC) from anonymous donor subjects. The prolyl hydroxylase inhibitor used for generating STEM CAR-T in this figure is 1,4-DPCA-ethyl-ester. As demonstrated in FIG. 8, over prolonged treatment of 6 co-culture cycles, the STEM CAR-T (ROR1) cells still show a higher secretion of cytokines such as interferon gamma (IFN-γ), interleukin 2 (IL-2), and tumor necrosis factor α (TNFα) compared to conventional CAR-T cells. At 6^{th} cycle of co-culture, STEM CAR-T shows about a two-fold enhancement in cytotoxicity towards triple negative breast cancer (TNBC) cells, demonstrating effective anti-tumor ability without reduction in efficacy due to exhaustion of T cells.

FIG. 9 shows increased pro-inflammatory cytokines secretion and lysis against antigen expressing cancer cells and delayed exhaustion status in HER2 targeting STEM CAR-T cells compared to conventional CAR-T. The T-cells used for generating CAR-T or STEM CAR-T are obtained from peripheral blood mononuclear cells (PBMC) from anonymous donor subjects. The prolyl hydroxylase inhibitor used for generating STEM CAR-T in this figure is 1,4-DPCA-ethyl-ester. As demonstrated in FIG. 9, over prolonged treatment of 6 co-culture cycles, the STEM CAR-T (HER2) cells still show a higher secretion of cytokines such as interferon gamma (IFN-γ), interleukin 2 (IL-2), and tumor necrosis factor α (TNFα) compared to conventional CAR-T cells. At 5^{th} cycle of co-culture, STEM CAR-T shows about doubled cytotoxicity towards triple negative breast cancer (TNBC) cells, demonstrating effective anti-tumor ability without reduction in efficacy due to exhaustion of T cells.

FIG. 10 provides ex vivo T cell viability data for exemplary prolyl hydroxylase inhibitors, 1,4-DPCA, 1,4-DPCA ethyl ester, Molidustat, Dencichine, Enarodustat, Roxadustat, IOX2 and Daprodustat. The T cells are activated in CD3/28 antibody in a cell culture medium with the addition of different prolyl hydroxylase inhibitors for 5 days. The T-cells used for generating CAR-T or STEM CAR-T are obtained from peripheral blood mononuclear cells (PBMC) from anonymous donor subjects. The antibody and inhibitor are removed from the cell culture medium from day 5 onwards. As shown in FIG. 10, all prolyl hydroxylase inhibitors show an increase in number of viable T cell relative to the number of total viable T cells of the negative control (DMSO) group, indicating improved T cell viability with treatment of prolyl hydroxylase inhibitors. The T cell proliferation rate for each prolyl hydroxylase inhibitor is calculated based on the number of viable T-cell relative to the number viable T-cell in the DMSO-treated group. Thus, it can be seen that prolyl hydroxylase inhibitors treated T cells show increased T cell viability.

FIG. 11 shows ex vivo CAR-T cell count for STEM CAR-T cells targeting ROR1/HER2/EGFR/CD19, respectively, compared to conventional CAR-T. The T-cells used for generating CAR-T or STEM CAR-T are obtained from peripheral blood mononuclear cells (PBMC) from anonymous donor subjects. To generate CAR T cells, bulk PBMCs were activated on day 0 using CD3 and CD28. A prolyl hydroxylase inhibitor, in this case 1,4-DPCA ethyl ester or an equivalent volume of DMSO was added to the cell culture on day 0. On day 3, cells were transduced with the lentivirus expressing the respective CAR. The activation lasted for 5 days and the CAR-T cells were counted manually against the percentage of CAR transduced as measured by Fluorescence-activated Cell Sorting (FACS). The total number of CAR-T cells are counted manually after CD3/28 antibody activation and the presence or absence of prolyl hydroxylase inhibitor (STEM CAR-T and conventional CAR-T, respectively) on days indicated on the graphs. As can be seen from FIG. 11, enhanced CAR-T cell expansion is observed in all STEM CAR-T cells compared to conventional CAR-T cells.

FIG. 12 provides in vivo tumor volume in different xenograft cancer models in mice after administration with CAR-T cells obtained using conventional methods or STEM modification method as disclosed herein. The T-cells used for generating CAR-T or STEM CAR-T are obtained from peripheral blood mononuclear cells (PBMC) from anonymous donor subjects. FIG. 12A shows the tumor volume over time in DLD-1 xenograft model for colorectal cancer upon treatment with CAR-T cells targeting EGFR. As can be seen from the graph, a significant reduction of tumor size is observed in CAR-T treated groups compared to phosphate buffered saline (PBS)-treated vehicle control. In particular, the CAR-T cells produced using STEM method as described herein show the lowest tumor volume within 16 days post injection. FIG. 12B shows the tumor volume over time in DLD-1 xenograft model for colorectal cancer upon treatment with CAR-T cells targeting HER2. As can be seen from the graph, significant reduction of tumor size is observed in STEM CAR-T treated groups compared to the conventional CAR-T group and the PBS-treated vehicle control group 16 days after injection. FIG. 12C shows the tumor volume over time in MB361 xenograft model for breast cancer upon treatment with CAR-T cells targeting HER2. As can be seen from the graph, significant reduction of tumor size is observed in STEM CAR-T treated groups compared to the conventional CAR-T group and the PBS-treated vehicle control group 16 days after injection. Therefore, the exemplary cancer disease models demonstrate that the STEM method as described herein increases CAR-T cell anti-tumor activity in vivo.

FIG. 13 profiles the CAR-T cells in xenograft model for colorectal cancer post administration. The T-cells used for generating CAR-T or STEM CAR-T are obtained from peripheral blood mononuclear cells (PBMC) from anonymous donor subjects. The circulating CAR-T cells in the blood are quantified. As shown in FIG. 13A, the number of EGFR targeting STEM CAR-T cells are highest compared to the PBS-treated vehicle control group and the conventional CAR-T cell-treated group upon administration in DLD-1 xenograft colorectal cancer model. FIG. 13B provides the population percentage of the number of T_{cm} and T_{scm} among total CAR-T cells produced using conventional CAR-T method, and STEM T method in vivo, in DLD-1 colorectal cancer model. The percentage of Tom and T_{scm} population was measured from the blood of the mouse taken 13 days post-CAR-T cells injection. The significant increase in T_{scm} and Tₑₘ population indicates increased stemness of STEM CAR-T cells. As shown in FIG. 13C, the number of HER2 targeting STEM CAR-T cells are highest compared to the PBS-treated vehicle control and the conventional CAR-T cell groups upon administration in the MDA-MB-361 breast cancer mouse model. FIG. 13D provides the population percentage of the number of Tₑₘ and T_{scm} among total CAR-T cells produced using conventional CAR-T method, and STEM T method in vivo, in MDA-MB-361 model breast cancer model. The percentage of Tom and T_{scm} population was measured from the blood of the mouse taken 13 days post-CAR-T cells injection. The significant increase in T_{scm} and T_{cm} populations indicates increased stemness of STEM CAR-T cells. Therefore, as demonstrated herein, STEM CAR-T cells obtained using the STEM method as described herein are present in large number in blood circulation even 2 weeks after administration. The STEM CAR-T cells show improved stemness compared to conventional CAR-T cells in vivo.

FIG. 14 shows the in vivo CAR-T cell count, tumor volume, and intratumoral CAR-T cell percentages in immune resistant triple negative breast cancer (TNBC) breast tumor mouse model MB231-LN treated with STEM CAR-T cells and conventional CAR-T cells. The T-cells used for generating CAR-T or STEM CAR-T are obtained from peripheral blood mononuclear cells (PBMC) from anonymous donor subjects. According to FIG. 14A, when treated with STEM CAR T-cells, the volume of the tumor grows much slower compared to the conventional CAR-T cells-treated and the PBS-treated vehicle control groups 19 days after the injection. The number of circulating CAR-T cells counted for the STEM CAR-T group is also significantly higher than the other two groups. FIG. 14B profiles the intratumoral CAR-T cell subtypes. The percentage of CAR-T cells infiltrating the tumor mass is quantified for STEM CAR-T (ROR1) and conventional CAR-T. A much higher infiltration of CAR-T cells is identified for the cells obtained using the STEM method as described herein, demonstrating improved tumor infiltration ability of STEM CAR-T cells. Among the tumor infiltrating CAR-T cells, the population of central memory CAR-T cells (T_{cm}) (CD45RO+CCR7+) shows an increase for the STEM CAR-T group. Administration of STEM CAR-T cells also show a small increase in the cytokine IFNγ+ population of CAR-T cell within tumor. Therefore, the STEM T method as described herein renders stronger anti-cancer activity for CAR-T cells and increases CAR-T cells tumor infiltration in solid tumor tissue.

FIG. 15 shows the in vivo CAR-T cell count, tumor volume, and probability of survival in DLD-1 colorectal cancer xenograft mouse model treated with exemplary STEM CAR-T cells compared to conventional CAR-T cells. The T-cells used for generating CAR-T or STEM CAR-T are obtained from peripheral blood mononuclear cells (PBMC) from anonymous donor subjects. The data in this figure was obtained from the same mice. As shown in FIG. 15A, the growth of the tumor has been reduced upon injection of STEM CAR-T (EGFR1-targeting) and the suppression effect is long lasting for over 50 days. FIG. 15B shows the number of CAR-T cells circulating in blood obtained from the mice treated with PBS, conventional CAR-T cells and STEM CAR-T cells. STEM CAR-T cells-treated mice exhibit a higher number of cells in blood compared to conventional CAR-T cells and the negative control over a prolonged period of time. FIG. 15C provides a survival curve for DLD-1 colorectal cancer xenograft mouse model treated with the CAR-T cells. Among the three groups, only STEM CAR-T cells-injected mice show high probably of survival over 80 days post injection. To conclude, STEM-CAR-T-treated mice showed long-lasting CAR-T cells in the circulation, suppressing the tumor from growth, and translating the durable immunity to higher survival rate in the treated subject. Therefore, T cells modified using the STEM method as described herein demonstrate superior anti-tumor characteristics compared to the non-modified T cells.

### DEFINITION

As used herein, the term “small molecule” refers to any organic compound with low molecular weight that may regulate a biological process. Many drugs are small molecules. The small size of the molecule allows it to enter cells easily, and are therefore often used as drugs targeting cellular proteins to affect molecular pathways. Conventionally, small molecule drugs have a size on the order of about 1 nm, or a molecular weight below about 500 Da.

As used herein, the term “T cell” or “T lymphocytes” refers to an important type of white blood cell and play a central role in the adaptive immune response. T cells are differentiated from hematopoietic stem cells, which are stem cells in the bone marrow. T cells can be distinguished from other lymphocytes by the presence of a T-cell receptor (TCR) on their cell surface. There are two major types of T cells: the CD4+ T cells (“helper T cells”) and the CD8+ T cells (“cytotoxic T cells”, or “killer T cells”). The CD8+ T cells are able to directly kill virus-infected cells, as well as cancer cells, and utilises cytokines to recruit other types of cells when mounting an immune response. Unlike the CD8+ killer T cells, the CD4+ cells function by further activating memory B cells and cytotoxic T cells, which leads to a larger immune response.

As used herein, the term “naïve T cells” or “Tₙ” refers to the immature T cells that have differentiated in the thymus. After the encounter with its cognate antigen within the periphery, a naïve T cell will be matured. The differentiation and activation of T cells is dependent on signals transduced by three different receptors: TCRs (including the CD4 and CD8 receptors that respond to MHC-II displayed antigens and MHC-I displayed antigens, respectively), costimulatory receptors, and cytokine receptors. These signals drive naïve T cells to differentiate into effector T cells or memory T cells.

As used herein, the term “effector T cells” or “T_{eff}” refers to a subset of T lymphocytes that have a relatively short lifespan. Effector T cells actively respond to a stimulus and carry out the functions of an immune response. Effector T cells can be cytotoxic T cells (CD8+), helper T cells (CD4+), and regulatory T cells (T_{reg}).

As used herein, the term “memory T cells” refers to a subset of T lymphocytes that are capable of mediating a faster and more potent immune response upon encounter with antigens they have prior exposure to. These cells are long-lived and can quickly expand to large numbers of effector T cells to protect against subsequent exposure to the same antigen. Memory T cells can be CD4+ cells, or CD8+ cells, depending on the type of antigen encountered.

Memory T cells comprise several subtypes. In general, the memory T cells include stem cell memory T (T_{scm}) cells and central memory T (T_{cm}) cells, which have different specific phenotypes and functions.

As used herein, the term “central memory T cells” or “T_{cm}” refers to one subtype of the memory T cells which express L-selectin, CD45RO and CCR7, and provide central immunosurveillance by patrolling the lymph nodes draining peripheral tissue sites in the body. Central memory T cells have several attributes in common with stem cells, the most important being the ability of self-renewal, mainly because of high level of phosphorylation on key transcription factor STAT5.

As used herein, the term “effector memory T cells” or “Tₑₘ” refers to another subtype of the memory T cells which express CD45RO but lack expression of CCR7 and L-selectin. Due to the lack the CCR7 lymph node-homing receptors, unlike central memory T cells, effector memory T cells are found in the peripheral circulation and tissues. Effector memory T cells are primarily active as the CD8 variants, thus being mainly responsible for cytotoxic action against pathogens. Tₑₘ cells express higher levels of receptors responsible for migration to inflamed tissues and have a stronger immediate effector function than T_{cm} cells.

As used herein, the term “stem cell-like memory T cells”, “stem memory T cells” or “T_{scm}” refer to another subtype of the memory T cells which express increased levels of CD95, IL-2Rβ, CXCR3, and LFA-1 compared with naïve T cell. Like naive T cells, T_{scm} cells are CD45RO−, CCR7+, CD45RA+, CD62L+ (L-selectin), CD27+, CD28+, and IL-7Rα+. Stem cell-like memory T cells exhibit characteristics of long lifespan, consistent self-renewing, rapid differentiation into effector T cells, and apoptosis resistance.

As used herein, the term “iPSC” or “induced pluripotent stem cells” refers to pluripotent stem cells derived from skin or blood cells, for example, that have been reprogrammed back into an embryonic-like pluripotent state that enables the development of an unlimited source of any type of human cell. As disclosed herein, the term “iPSC induced T cells”, “iPSC derived T cells”, or “T-iPSCs” refers to T cells that are re-differentiated from induced pluripotent stem cells. iPSC-derived T cells are phenotypically defined, expandable, and as functional as physiological T cells.

As used herein, the term “immunotherapy” refers to a method of treatment or prevention of disease by stimulation of the immune system to activate or suppress an immune response. The term “cellular immunotherapy”, or “adoptive cell therapy” refers to a type of immunotherapy in which patients' own immune cells are given to the patients to help the body fight diseases such as cancer. The immune cells can be expanded ex vivo to improve in the total number of cells, or are engineered to target specific tumor cell types. The term “adoptive T cell immunotherapy therapy” refers to an adoptive cell therapy that utilises T cells.

As used herein, the term “CAR” or “chimeric-antigen-receptor” refers to a recombinant receptor for antigens which redirect the specificity and function of T lymphocytes and/or other immune cells in a single molecule such that they are programmed to target tumor-associated antigens. Chimeric antigen receptors (CARs) usually consist of an extracellular domain that binds to a specific antigen on tumor cells, a transmembrane domain and intracellular domains that provide signals for T cell activation to attack tumor cells.

As used herein, the term “CAR-T cells” or “chimeric antibody receptor engineered T cell” refers to engineered T cells that express cancer specific artificial chimeric-antigen-receptor (CAR), which can be used in an adoptive T cell immunotherapy therapy. The T cells are obtained from patient's blood and are produced ex vivo. Large number of CAR-T cells are given to the patient by infusion to treat diseases such as cancer.

As used herein, the term “TCR-T cells” or “T cell receptor (TCR) T cells” refers to T cell receptor (TCR)-engineered T cells that are directed to target against specific tumor markers. Both CAR-T and TCR-T cell therapies improve the ability of T cell receptors to recognize and attack specific antigenic cell antigens by means of genetic modification. CAR-T directly changes one part of the T cell receptor into a specific antibody, allowing T cells to directly attack cancer cells under the guidance of antibodies. In contrast to CAR-T which recognizes surface antigens, TCR-T is capable of interacting with peptide-major histocompatibility complex (pMHC) generated from intracellular antigen proteolysis. TCR-T recognises a broad range of cancers while CAR-T offers more specific and potent targeting of tumor cells.

The production of CAR-T cells requires several carefully performed steps, and quality control testing is performed throughout the entire protocol. Briefly, the conventional CAR-T cell production process/method as referred to herein involves: (1) isolating leukocytes from the subject; (2) enrichment of T cells from the leukocytes; (3) activation of T cells and co-incubation of the T cells with viral vector encoding the CAR; and (4) expansion of CAR-T cells and final formulation.

As used herein, the term “T cell activation” refers to the process by which an antigen-presenting cell (APC) activates a T cell. Methods of activating T cells for the purpose of immunotherapy are known in the art. For example, autologous antigen-presenting cells (APCs) can be isolated and purified from the patient for T cell activation. Anti-CD3 antibodies can be used alone or in combination with feeder cells and growth factors, such as IL-2, which has been commonly used by a person skilled in the art. Alternatively, to simplify and standardise the activation process with higher efficiency, beads coated with anti-CD3/anti-CD28 monoclonal antibodies can be used.

As used herein, the term “cancer” or “malignancy” refers to a large group of diseases that can start in almost any organ or tissue of the body when abnormal cells grow uncontrollably and go beyond their usual boundaries to invade adjoining parts of the body and/or spread to other organs.

As used herein, the term “tumor” is a collection of cells/tissues grown in a lump due to the uncontrollable multiplication of abnormal or damaged cells. Tumors can be cancerous or non-cancerous (benign). Cancerous tumors spread into, or invade, nearby tissues and can travel to distant places in the body to form new tumors (metastasis). As used herein, the term “solid tumor” refers to one group of tumor that usually does not contain cysts or liquid areas. Examples of solid tumors are sarcomas, carcinomas, and lymphomas.

As used herein, the term “differentiation” refers to the process in which a stem cell changes from a less specialized cell type to a more specialized type, involving a switch from proliferation to specialization. Differentiation changes a cell's size, shape, membrane potential, metabolic activity, and responsiveness to signals. These changes are largely due to highly controlled modifications in gene expression and are the study of epigenetics.

As used herein, the term “stem cell” refers to a cell with the potential to self-renew and to develop into many different specialised functional types of cells in the body (differentiation). As used herein, the term “stemness” refers to the ability for a cell to self-renewal and differentiate. Depending on their ability to differentiate, stem cells can be categorized into the following groups: (1) totipotent stem cells: cells that can differentiate into all cell types; (2) pluripotent stem cells: cells that can differentiate into almost all cell types; (3) multipotent stem cells: cells that can differentiate into a related family of cell types; (4) oligopotent stem cells: cells that can differentiate into a few different cells; (5) unipotent stem cells: cells that can produce one cell type only.

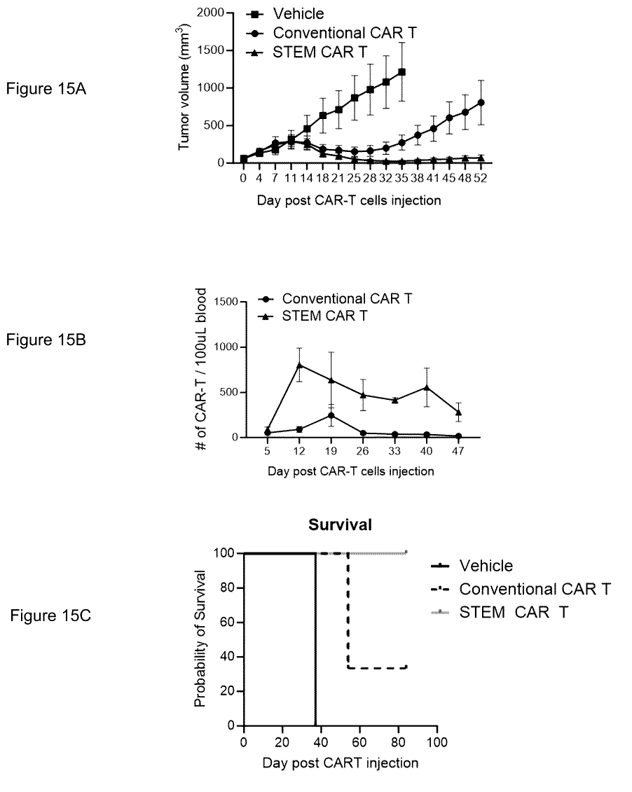

As used herein, the term “memory” refers to the population of memory T cells among the total T cell population. Therefore, the expression “enrichment of memory” or “improved memory” described the increase in population percentage of memory T cells such as stem cell memory T (T_{scm}) cells, central memory T (T_{cm}) cells, and effector memory T (Tₑₘ) cells. As exemplified in FIGS. 14, 15, and 24, STEM CAR-T cells show an overall T cell memory enrichment characterised by the increase in the population percentage of T_{scm} and Tₑₘ cells compared to conventional CAR-T cells.

As used herein, the term “exhaustion” refers to a dysfunctional and hyporesponsive cellular state commonly observed in response to persistent antigen exposure, for example, in chronic infection. In the context of the present disclosure, “T cell exhaustion” refers to such state associated with T cells characterised by progressive loss of T cell effector functions and self-renewal capacity in tumor microenvironment, thereby limiting the efficacy of immunotherapy. Therefore, T cell exhaustion is often associated with poor tumor control in patients.

As used herein, the term “prolyl hydroxylase”, “procollagen-proline dioxygenase”, or “prolyl 4-hydroxylase” refers to a member of the class of enzymes known as alpha-ketoglutarate-dependent hydroxylases (EC number: 1.14.11.2). Prolyl hydroxylase catalyses proline residues from diverse protein substrates irreversibly to (2S,4R)-4-hydroxyproline (Hyp). Such hydroxylation reaction is the most common post-translational modification in humans.

As used herein, the term “prolyl hydroxylase inhibitor” refers to agents that inhibit the enzymatic activity directly or indirectly. For example, the prolyl hydroxylase inhibitors can be, but are not limited to

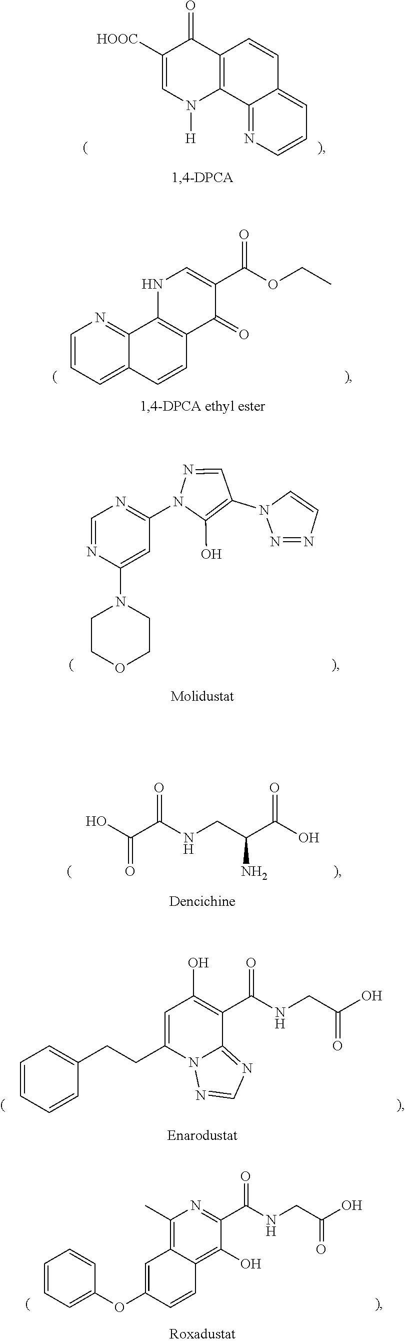 [SMILES: O=C(O)c1c[nH]c2c(ccc3cccnc32)c1=O ; CCOC(=O)c1c[nH]c2c(ccc3cccnc32)c1=O ; Oc1c(-n2ccnn2)cnn1-c1cc(N2CCOCC2)ncn1 ; N[C@@H](CNC(=O)C(=O)O)C(=O)O ; O=C(O)CNC(=O)c1c(O)cc(CCc2ccccc2)n2ncnc12 ; Cc1nc(C(=O)NCC(=O)O)c(O)c2ccc(Oc3ccccc3)cc12]

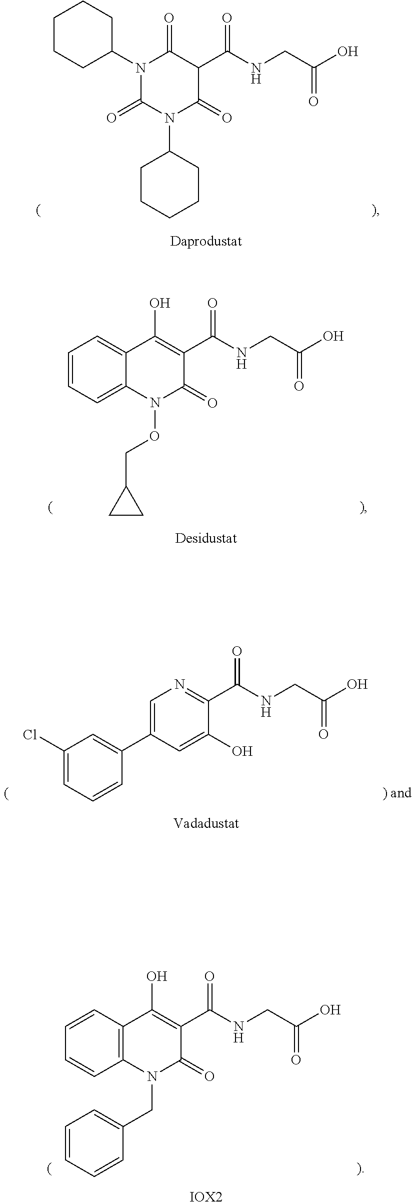

As used herein, the term “triple negative breast cancer” or “TNBC” refers to a breast cancer, which had been tested and found to lack (or be negative) for human epidermal growth factor receptor 2 (HER-2), estrogen receptors (ER), and progesterone receptors (PR). Triple negative cancers are also known to be called “basal-like” cancers.

As used herein, the term “reprogramming” refers to the process that set somatic cell fate and restoration of a cell to the pluripotent state for reconstruction of cell fate. As used herein, “T cell reprogramming” refers to the process of altering the function, phenotype, or differentiation state of T cells to enhance their therapeutic potential.

As used herein, the term “modification” refers to the process as disclosed herein which induces changes in the characteristics of T cells. The changes include, for example, increasing the expansion rate of modified T cells compared to unmodified T cells, improving ex vivo stemness of modified T cells compared to unmodified T cells, increasing population of T memory cell in the modified T cells compared to unmodified T cells, increasing antigen specific cytotoxicity of modified T cells compared to unmodified T cells, and reducing exhaustion of modified T cells compared to unmodified T cells.

### DETAILED DESCRIPTION

The present disclosure provides a small molecule-based T cell modification method to induce T cell reprogramming, leading to increase in T cell stemness and enrichment of memory T cell populations. The method described herein applies to T cells in general, and can be incorporated with existing T-cell based cellular therapies such as CAR-T or TCR-TCR-T. The methods described herein effectively shorten the ex vivo manufacturing timeline. The resulting modified T cells show durable anti-tumor effects and improved proliferation and viability of T cells in vivo. The modification methods as described herein has the potential to overcome the current hurdles of cellular immunotherapy in solid tumors, which includes the lack of in vivo persistence and durable anti-tumor response due to fast T cell exhaustion in the tumor microenvironment.

Therefore, in one aspect, the present disclosure provides a method of modifying T cells. In one example, the method of modifying T cells is an in vitro or ex vivo method. In another example, the method described herein can take place during T cell activation. In some examples, the method comprises contacting the T cells with an inhibitory agent for prolyl hydroxylase.

In one example, the time period for modifying the T cells can be about 1 day, about 3 days, about 4 days, about 5 days, about 6 days, and about 7 days. In some further examples, the duration of modifying the T cells can be about 1 to 5 days, about 2 to 5 days, about 3 to 5 days, about 4 to 5 days, about 2 to 3 days, about 2 to 4 days, about 3 to 4 days, about 2 to 6 days, about 1 to 7 days, about 2 to 7 days, about 3 to 6 days, about 3 to 7 days, about 4 to 6 days, about 4 to 7 days, about 5 to 6 days, and about 5 to 7 days. In one example, the duration of modifying the T cells can be about 1 to 5 days. In another example, the duration of modifying the T cells can be less than a week.

As used herein, the term “inhibitory agent” generally refers to an agent that directly or indirectly slows, suppresses, or interferes with the enzymatic activity of prolyl hydroxylase. The inhibitory agent can include, but is not limited to: a compound, a small molecule drug, an enzyme, an antibody, a nucleic acid, a protein, a polymer, or a combination thereof. As used herein, the term “prolyl hydroxylase”, “procollagen-proline dioxygenase”, or “prolyl 4-hydroxylase” refers to a member of the class of enzymes known as alpha-ketoglutarate-dependent hydroxylases (EC number: 1.14.11.2). Prolyl hydroxylase catalyses proline residues from diverse protein substrates irreversibly to (2S,4R)-4-hydroxyproline (Hyp). Such hydroxylation reaction is the most common post-translational modification in humans. Therefore, a person skilled in the art would appreciate that an agent that, regardless of its mechanism of action, inhibits or reduces the hydroxylation activity of prolyl hydroxylase, would be suitable for the purpose as described herein.

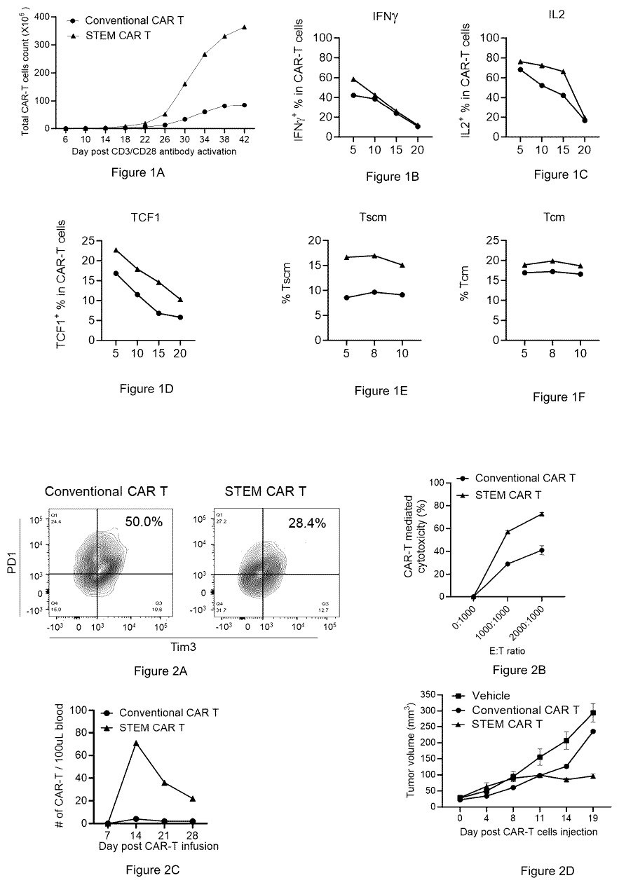

In some examples, the inhibitory agent for prolyl hydroxylase is a small molecule prolyl hydroxylase inhibitor. As exemplified in FIG. 1, the small molecule prolyl hydroxylase inhibitor can be comprised in a cell culture medium, such as a T cell culture medium to allow contacting of the inhibitory agent for prolyl hydroxylase with T cells. Apart from the examples provided herein, other ways of contacting an inhibitory agent with T cells are well known in the art.

In some further examples, the inhibitory agent for prolyl hydroxylase is a small molecule inhibitor. Conventionally, small molecule drugs have a size on the order of about 1 nm, or a molecular weight below about 500 Da. Small molecule inhibitor drugs for prolyl hydroxylase have been actively developed due to its clinical relevance for the treatment of diseases such as chronic kidney diseases. For example, the prolyl hydroxylase inhibitors can be, but are not limited to:

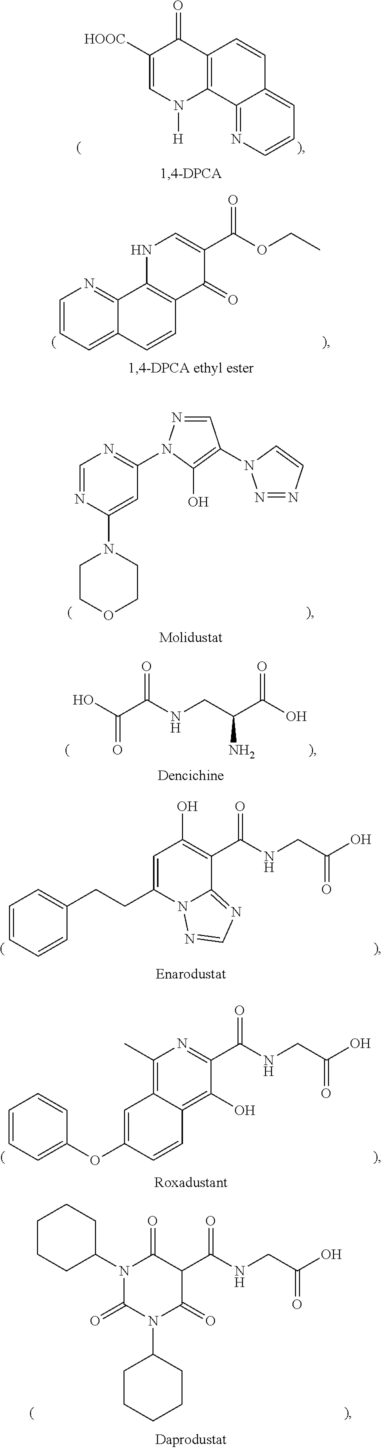

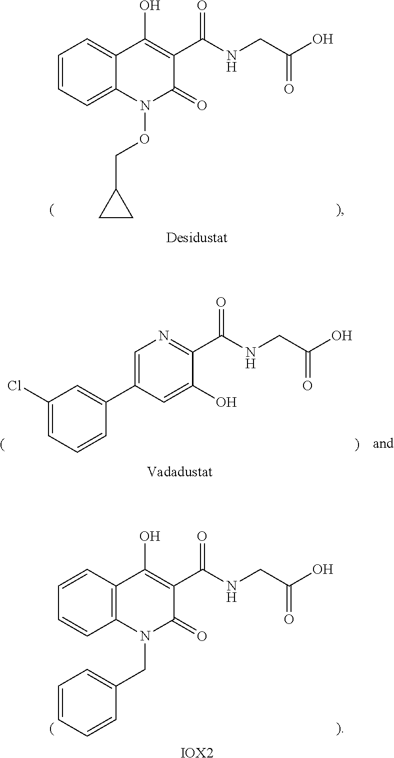

In one example, the small molecule inhibitor for prolyl hydroxylase is 1,4-DPCA-ester

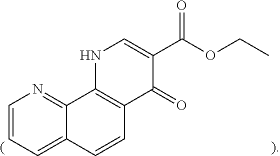 [SMILES: CCOC(=O)c1c[nH]c2c(ccc3cccnc32)c1=O]

As described herein, for example, the concentration of the small molecule prolyl hydroxylase inhibitor can be about 0.5 μM, about 0.75 μM, about 1 μM, about 1.25 μM, about 1.5 μM, about 1.75 μM, about 2 μM, about 2.25 μM, about 2.5 μM, about 2.75 μM, or about 3 μM. In some examples, the concentration ranges of the small molecule prolyl hydroxylase inhibitor can be about 1.25 μM to about 1.75 μM, about 1 μM to about 2 μM, about 0.75 μM to about 2.25 μM, about 0.5 μM to about 2.5 μM, about 0.25 μM to about 2.75 μM. In one example, the concentration of the small molecule prolyl hydroxylase inhibitor is about 1 μM. Apart from the exemplary concentrations and concentration ranges provided herein, it is within the capability for a person skilled in the art to determine a suitable amount of the small molecule prolyl hydroxylase inhibitor based on the characteristics of the inhibitor chosen, with considerations of the teaching of the present disclosure and the condition of the cells.

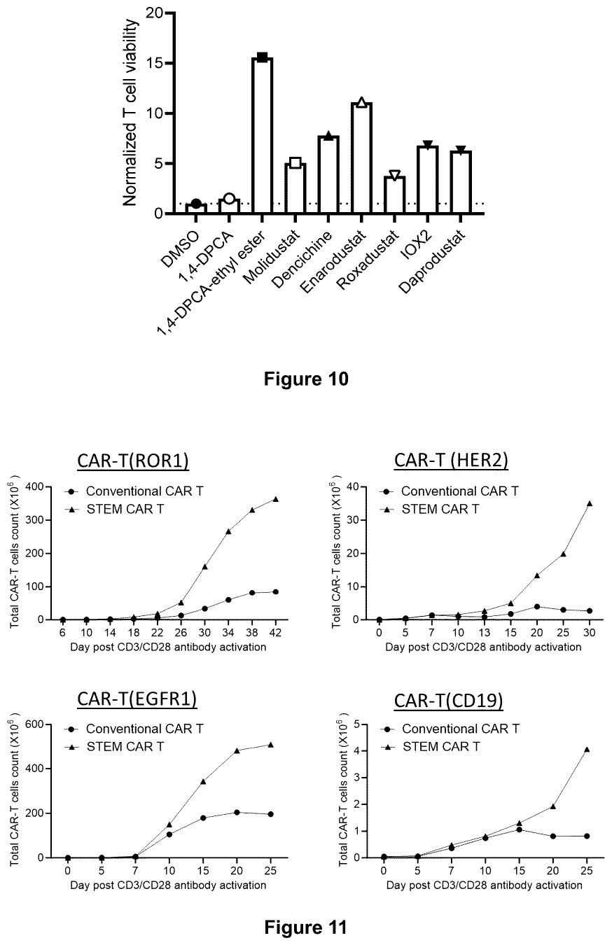

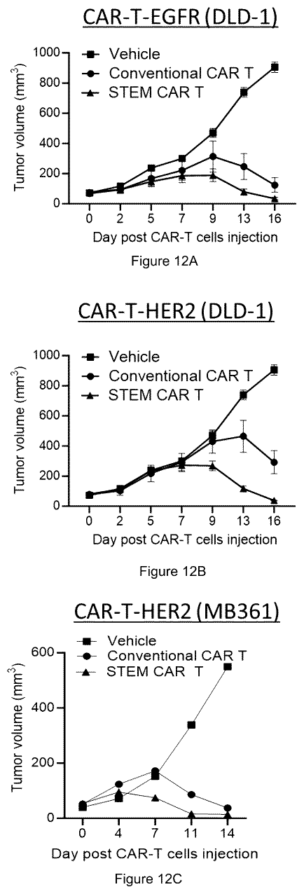

In another aspect, the present disclosure provides a method of preparing modified T cells. The method as described herein comprises modifying T cells during T cell activation by contacting the T cells with an inhibitory agent for prolyl hydroxylase. In one example, the modifying comprises increasing the expansion rate of T cells, compared to unmodified T cells. For example, as shown in FIG. 11, the ex vivo proliferation rate of isolated T cells is measured after treatment with an inhibitory agent for prolyl hydroxylase. All treated T cells expressing different CAR show increased proliferation compared to the control group, which is the conventional CAR-T cells. The viability of T cells treated with eight exemplary inhibitory agents for prolyl hydroxylase also shows significant increase, according to FIG. 10, which contributes to the increase in expansion rate of T cells as well.

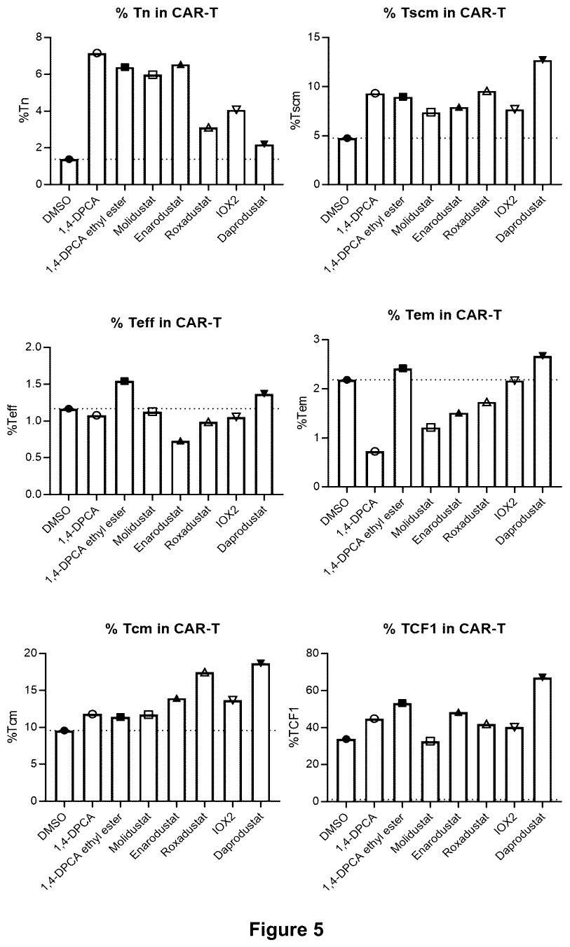

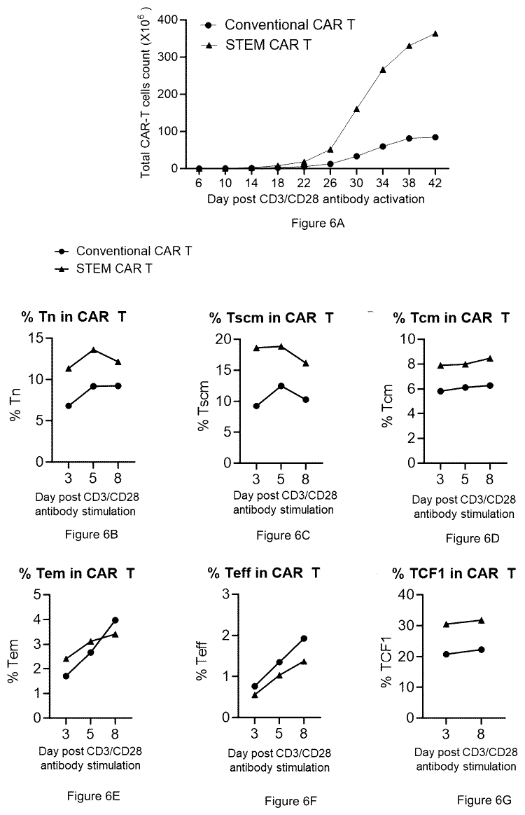

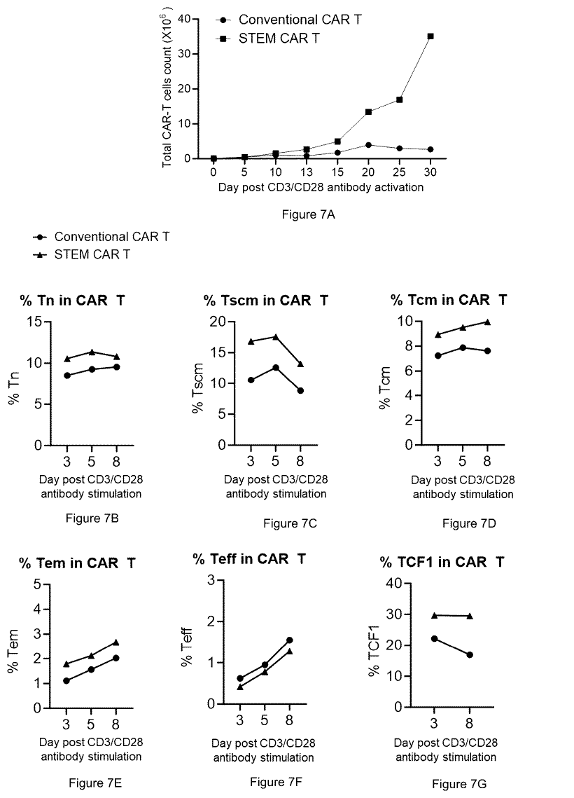

In another example, the modified T cells comprise an increasing population of memory T cell among the total T cell population, compared to unmodified T cells. As shown in FIG. 5, for example, after treatment with seven exemplary inhibitory agents for prolyl hydroxylase, the populations of Naïve T cells (Tₙ), stem-like memory T-cell (T_{scm}) and central memory T cell (T_{cm}) are higher than DMSO-treated control group 12 days post treatment. In another example, modifying T cells comprises improving the ex vivo T cell stemness, compared to unmodified T cells. As used herein, stemness refers to the ability for a cell to self-renewal and differentiate. As shown in FIG. 5, for example, after treatment with seven exemplary inhibitory agents for prolyl hydroxylase, the TCF1 expression is higher than DMSO at D5 and D8 post treatment.

In another example, the modifying of T cells comprises increasing antigen specific cytotoxicity of the modified T cells, compared to unmodified T cells. As shown in FIG. 2B, for example, which provides a comparison between modified STEM CAR-T cells and conventional unmodified CAR-T cells in mediating cytotoxicity in cancer cells. Modified cells show almost doubled cytotoxicity compared to unmodified ones, and the effect is proportional to the increase of effector (E) to target (T) ratio. Therefore, the modification method as disclosed herein provides effective improvement in cytotoxicity against target cells, such as cancer cells.

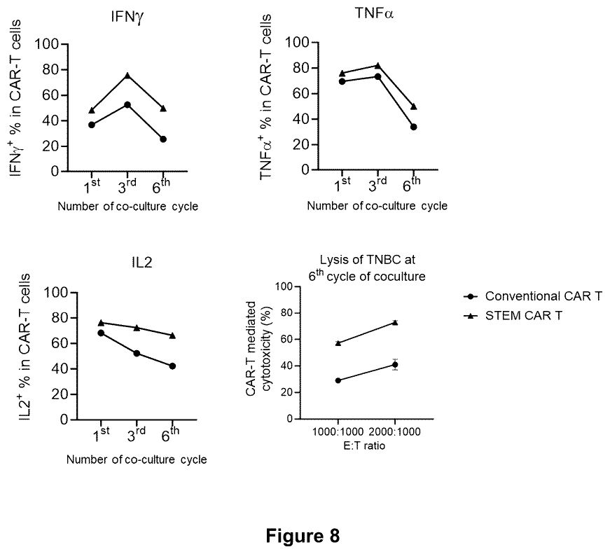

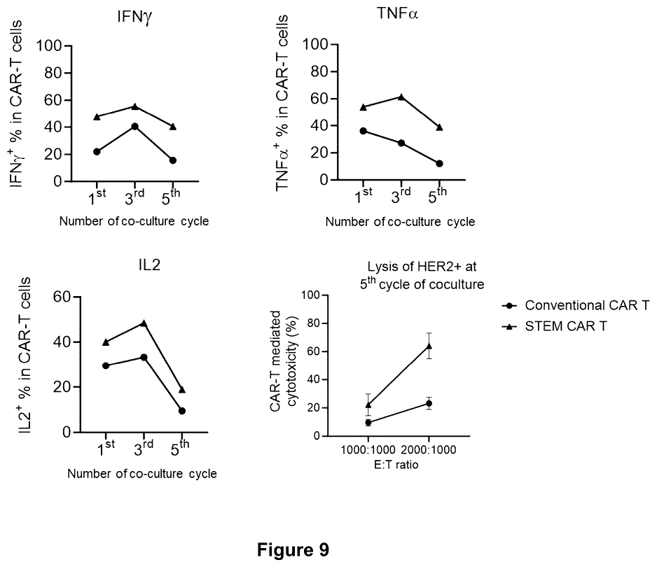

In another example, the modified T cells comprises reducing T cell exhaustion in vivo, compared to unmodified T cells. As demonstrated in FIG. 2A, ROR1 targeting CAR-T cells modified with the method as described herein (STEM CAR-T cells) show reduction of exhaustion markers such as PD1 and Tim3 after 6 co-cultures with tumor cells. FIGS. 8 and 9 provide additional examples of modified STEM CAR-T cells (targeting ROR1 and HER2, respectively). Both modified T cells targeting ROR1 and HER2 show increased pro-inflammatory cytokines secretion and lysis against antigen expressing cancer cells and delayed exhaustion status over prolonged treatment of 5 to 6 co-culture cycles. Therefore, the modification method as described herein reduces T cell exhaustion compared to unmodified T cells in vivo.

The method described herein applies to T cells in general, and can be incorporated with existing T-cell based cellular therapies. In one example, the modified T cells obtained using the method disclosed herein can be used for adoptive T cell immunotherapy. In another example, the modified T cells obtained using the method disclosed herein can be used for Chimeric Antigen Receptor (CAR) T cell therapy. In yet another example, the modified T cells obtained using the method disclosed herein can be used for T Cell Receptor (TCR) T cell therapy.

Therefore, in a further aspect, the present disclosure provides a method of producing Chimeric Antigen Receptor (CAR) T cells. In one example, the method comprises modifying T cells as disclosed herein. In another example, the method comprises modifying T cells during T cell activation by contacting the T cells with an inhibitory agent for prolyl hydroxylase. Methods of activating T cells are known in the art. For example, autologous antigen-presenting cells (APCs) can be isolated and purified from the patient for T cell activation. Anti-CD3 antibodies can be used alone or in combination with feeder cells and growth factors, such as IL-2, which have been commonly used by a person skilled in the art. In one example of the method as described herein, the T cell activation can be induced by anti-CD3 antibody and/or anti-CD28 antibody. Alternatively, to simplify and standardise the activation process with higher efficiency, beads coated with anti-CD3/anti-CD28 monoclonal antibodies can be used. In another example, other cytokines such as IL17 or IL15 can also be used.

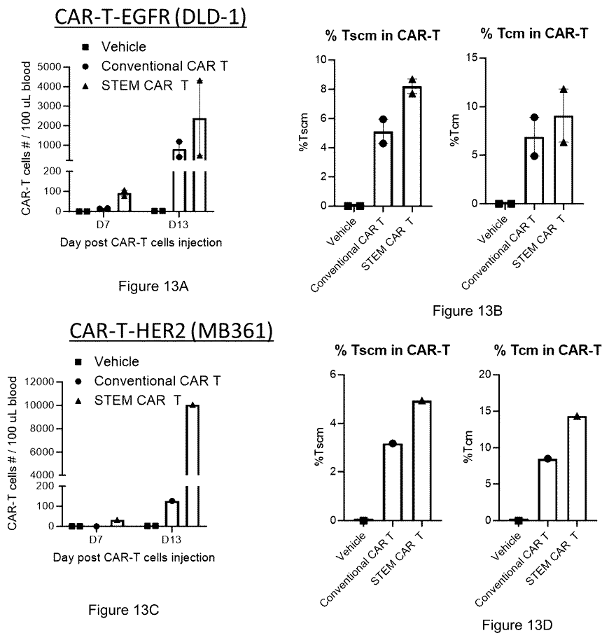

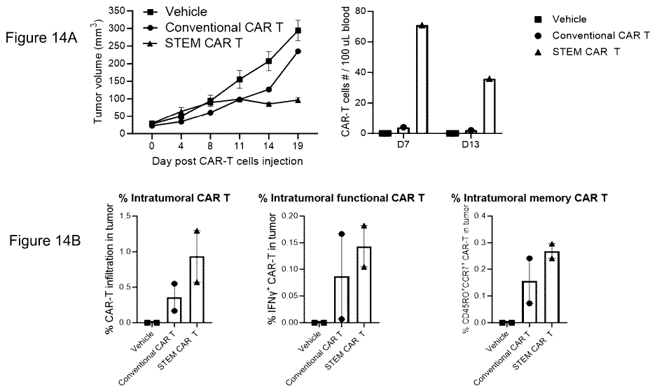

In another example, the method of producing Chimeric Antigen Receptor (CAR) T cells comprises modifying T cells during T cell activation by contacting the T cells with an inhibitory agent for prolyl hydroxylase, and introducing a CAR transgene into the modified T cells to produce CAR-T cells. Methods of designing a CAR transgene and methods of introducing a CAR transgene into T cells are known in the art. The CAR transgene comprises a target sequence that allows recognition and clearance of tumor cells by CAR-T cells. There are a variety of target sequences available for cellular immunotherapy, such as C-type lectin-like molecule-1 (CLL-1), CD19, CD20, B cell maturation antigen (BCMA), HER2, ROR1, and EGFP1. The present disclosure in FIG. 9, for example, provides modified STEM CAR-T cells targeting HER2 for treatment of breast cancer. The present disclosure in FIG. 13, for example, provides modified STEM CAR-T cells targeting EGFR for treatment of colorectal cancer. Based on the disease to be treated and the genotype of the tumor specifically targeted, a person skilled in the art would be able to design a suitable CAR transgene. The manufacture process for CAR-T cells involves, briefly: (1) isolating leukocytes from the subject; (2) enrichment of T cells from the leukocytes; (3) activation of T cells and co-incubation of the T cells with viral vector encoding the CAR. In one example, the CAR transgene is introduced into the modified T cells using a lentivirus. In another example, the steps of modifying the T cells and the step of introducing a CAR transgene are carried out simultaneously or sequentially. A person skilled in the art would be able to follow the teaching of the present disclosure and the available protocols for introducing a CAR transgene into the modified T cells as described herein.

In a further example, the method of producing Chimeric Antigen Receptor (CAR) T cells comprises modifying T cells during T cell activation by contacting the T cells with an inhibitory agent for prolyl hydroxylase, introducing a CAR transgene into the modified T cells to produce CAR-T cells, and harvesting the CAR-T cells obtained. In one example, the modifying T cells during T cell activation increases the number of CAR-T cells harvested. The harvested CAR-T cells can be preserved, for example, by cryopreservation, or infused into a patient in need.

For example, after 1-3 days of stimulation, a chimeric-antigen-receptor (CAR) cassette is introduced via lentivirus. The CAR-T cells are collected 2 days later for injection. The entire manufacturing process takes less than one week. The method utilizes the small molecule prolyl hydroxylase inhibitor compound at a low dose and for a relatively short term, thereby incurring minimum cost during manufacture and allowing an easy scale-up of standard operational procedures.

In another aspect, the present disclosure provides a method of producing T Cell Receptor (TCR) T cells. Like CAR-T cell therapy, engineered T cell receptor (TCR) therapy involves treating cancer cells with the patient's activated T lymphocytes. Both strategies give T cells new receptors to enable more effective targeting of cancer cells. In one example, the method comprises modifying T cells as disclosed herein. In another example, the method comprises modifying T cells during T cell activation by contacting the T cells with an inhibitory agent for prolyl hydroxylase. Methods of activating T cells are known in the art. For example, autologous antigen-presenting cells (APCs) can be isolated and purified from the patient for T cell activation. Anti-CD3 antibodies can be used alone or in combination with feeder cells and growth factors, such as IL-2, which has been commonly used by a person skilled in the art. In one example of the method as described herein, the T cell activation can be induced by anti-CD3 antibody and/or anti-CD28 antibody. Alternatively, to simplify and standardise the activation process with higher efficiency, beads coated with anti-CD3/anti-CD28 monoclonal antibodies can be used. In another example, other cytokines such as IL17 or IL15 can also be used.

In another example, the method of producing T Cell Receptor (TCR) T cells comprises modifying T cells during T cell activation by contacting the T cells with an inhibitory agent for prolyl hydroxylase, and introducing a TCR transgene into the modified T cells to produce TCR-T cells. Methods of designing a TCR transgene and methods of introducing a TCR transgene into T cells are known in the art. Similar to CAR-T, the TCR transgene comprises a target sequence that allows recognition and clearance of tumor cells by TCR-T cells. Based on the disease to be treated and the genotype of the tumor specifically targeted, a person skilled in the art would be able to design a suitable TCR transgene. A person skilled in the art would also be able to follow the teaching of the present disclosure and the available protocols for introducing a TCR transgene into the modified T cells as described herein.

In a further example, the method of producing T Cell Receptor (TCR) T cells comprises modifying T cells during T cell activation by contacting the T cells with an inhibitory agent for prolyl hydroxylase, introducing a TCR transgene into the modified T cells to produce TCR-T cells, and harvesting the TCR-T cells obtained. In one example, the modifying T cells during T cell activation increases the number of TCR-T cells harvested. The harvested TCR-T cells can be preserved, for example, by cryopreservation, or infused into a patient in need. In a further example, the harvested CAR-T or TCR-T cells can be used for adoptive T cell immunotherapy.

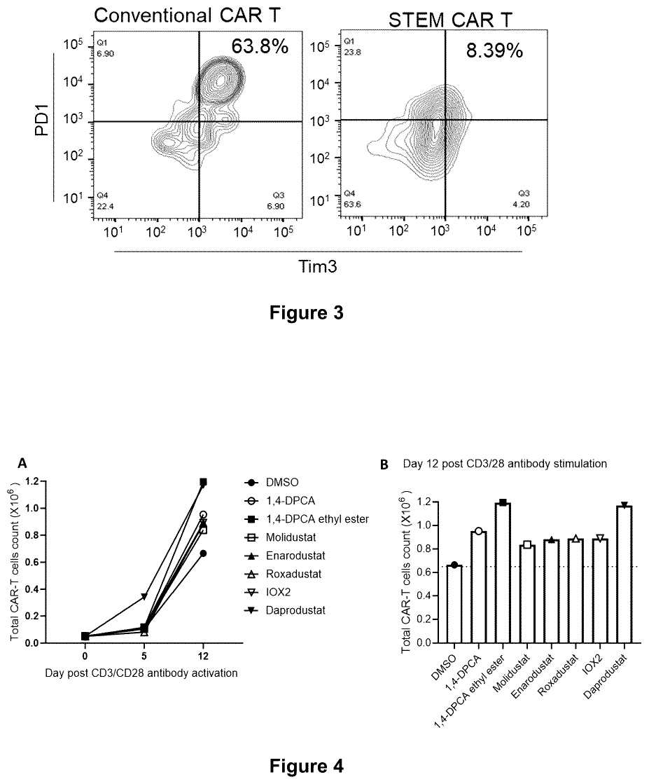

In some examples of the methods as described herein, the harvested CAR-T or TCR-T cells show reduced T cell exhaustion after repeated tumor antigen stimulations compared to unmodified T cells. T cell exhaustion refers to a condition in which T cells lose their ability to kill certain cells, such as cancer cells or cells infected with a virus over prolonged activation. Exhausted T cells in cancer show high expression levels of inhibitory receptors, such as PD-1, CTLA-4, TIM-3, LAG-3, BTLA and TIGIT, and reduced effector cytokine production, such as IL-2, TNF-α, IFN-γ and GzmB. The reduction of T cell exhaustion can be characterized by the changes in the expression levels of T cell exhaustion markers compared to unmodified T cells. In one example, the reduced T cell exhaustion is characterized by reduced expression of T cell exhaustion markers PD-1 and TIM-3 compared to unmodified T cells. In another example, the reduced T cell exhaustion is characterized by reduced upregulation of T cell exhaustion markers PD-1 and TIM-3 compared to unmodified T cells. As demonstrated in FIG. 2A and FIG. 3, for example, reduction of exhaustion markers are observed in STEM CAR-T cells after prolonged exposure to tumor cells.

In some examples of the methods as described herein, the harvested CAR-T or TCR-T cells show increased level of cytokine release compared to unmodified CAR-T or TCR-T cells. T cells as the major effector cells in cellular immunity, produce cytokines in immune responses to mediate inflammation and regulate other types of immune cells. The activation and proliferation of CAR-T and TCR-T cells release primary cytokines such as IL1, IFN-γ, and TNF, which induce the activation of other immune cells, such as macrophages, DCs, and monocytes. These cells then produce excessive amounts of secondary cytokines, such as IL6, IL10, and IL5. Cytokines can regulate the growth, apoptosis, activation, and differentiation of target cells. Significant correlations between the concentration of cytokines are reported with the prognosis of cancer patients. In one example, the cytokines released by the harvested CAR-T or TCR-T cells can be, but are not limited to IFN-γ, IL-2, antigen Ki67, and TNFα.

In the methods of the present disclosure, the T cells can be isolated T cells, derived from stem cells or iPSC induced T cells. In one example, the T cells are isolated from a sample from a subject. The subject can be a healthy subject, or a subject who receives cellular immunotherapy. The subject can be at risk of being diagnosed for having a cancer, or has been diagnosed for having a cancer, or is being treated for a cancer.

T cells can be obtained from blood, hematopoietic stem cell-derived lymphoid progenitor cells, embryonic stem cell (ESC), or induced pluripotent stem cell (iPSC)-derived T cells. The samples for T cell isolation can be, but are not limited to a blood sample, or a surgically removed tumor sample. In one example, the sample comprises peripheral blood mononuclear cell (PBMC).

In another aspect, the present disclosure provides a modified T cell obtained by the methods described herein. In another aspect, the present disclosure provides a CAR-T cell obtained by the methods described herein. In another aspect, the present disclosure provides a TCR-T cell obtained by the methods described herein. In a further aspect, the present disclosure provides the modified T cell as disclosed herein, the CAR-T cell as disclosed herein, or the TCR-T cell as disclosed herein for use in therapy.

In another aspect, the present disclosure provides a method of treating or alleviating a cancer in a subject. The treatment or alleviation outcome in the subject can be evaluated based on established clinical standards. For example, assessments can include but are not limited to: general function, quality of life (QOL), pain, cognition, fatigue, and objective measures such as tumor size and overall survival (OS) and progression-free survival (PFS).

In another aspect, the present disclosure provides a method of inducing long-term anti-tumor effect in a subject having a cancer.

In another aspect, the present disclosure provides a method of reducing tumor size in a subject having a cancer.

In another aspect, the present disclosure provides a method of increasing memory T cell and CAR-T cell stemness in vivo in a subject.

In another aspect, the present disclosure provides a method of increasing tumor infiltration of T cells in a subject having cancer.

In another aspect, the present disclosure provides a method of increasing the in vivo expansion rate of CAR-T cells or TCR-T cells in a subject.

The methods as described herein, comprise administering a therapeutically effective amount of the modified T cell as described herein, administering a therapeutically effective amount of a CAR-T cell as described herein, or administering a therapeutically effective amount of a TCR-T cell as described herein to the subject in need thereof. As used herein, the term “pharmaceutically effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with the disease state (e.g., relieving the infection). A person skilled in the art is able to determine a pharmaceutically effective amount for the CAR-T or the TCR-T cells as described herein, the composition, or the pharmaceutical composition as disclosed herein based on considerations such as disease state, body size, administration frequencies and route. Any pharmaceutical or medical composition described herein can be administered together with an acceptable pharmaceutical excipient and/or additive, and/or carrier. Such additional components are well know in the art.

The administration of the CAR-T cell or the TCR-T cell can be by intravenous infusion. A person skilled in the art would appreciate that other administration routes can be applicable for intact delivery of the cells to the subject. The administration can be a one-time (single dose) administration or a repeated administration. In one example, the CAR-T cell or the TCR-T cell is formulated as single units for administration. In another example, the CAR-T cell or the TCR-T cell is formulated as multiple units for administration, such as two, three or four units per day or per week. The amount and frequence of administration can be determined accordingly by a person skilled in the art based on the disease state and/or the subject's condition, for example.

In some examples, the modified cells, the CAR-T cell or the TCR-T cell as described herein can be used in combination with other agents. For example, an anti-cancer agent, or a therapeutic agent for the control of symptoms, or an agent to improve the delivery efficiency of the cells. In some examples, the modified cells, the CAR-T cell or the TCR-T cell as described herein can be used in combination with other anti-cancer therapies.

In another aspect, the present disclosure provides the use of the modified T cell as disclosed herein, the CAR-T cell as disclosed herein, or the TCR-T cell as disclosed herein in the manufacture of a medicament for treating a cancer. In another aspect, the present disclosure provides the use of the modified T cell as disclosed herein, the CAR-T cell as disclosed herein, or the TCR-T cell as disclosed herein in the manufacture of a medicament for inducing long-term anti-tumor effect in a subject having a cancer. In another aspect, the present disclosure provides the use of the modified T cell as disclosed herein, the CAR-T cell as disclosed herein, or the TCR-T cell as disclosed herein in the manufacture of a medicament for reducing tumor size in a subject having a cancer. In another aspect, the present disclosure provides the use of the modified T cell as disclosed herein, the CAR-T cell as disclosed herein, or the TCR-T cell as disclosed herein in the manufacture of a medicament for increasing memory T cell and CAR-T cell stemness in vivo in a subject. In another aspect, the present disclosure provides the use of the modified T cell as disclosed herein, the CAR-T cell as disclosed herein, or the TCR-T cell as disclosed herein in the manufacture of a medicament for increasing tumor infiltration of T cells in a subject having a cancer. In another aspect, the present disclosure provides the use of the modified T cell as disclosed herein, the CAR-T cell as disclosed herein, or the TCR-T cell as disclosed herein in the manufacture of a medicament for increasing the in vivo expansion rate of CAR-T cells or TCR-T cells in a subject.

In the methods or use as disclosed herein, the subject can have a cancer, or is at risk of having cancer. In some examples, the cancer is a benign or malignant cancer. Methods and clinical standards for determining whether a cancer is a benign or malignant cancer is known to a person skilled in the art. For example, the cancer can include, but is not limited to: leukemia, myeloma, sarcoma, melanoma, lymphoma, cancer of the breast, colon, bladder, prostate, lung, kidney, pancreas, liver, uterus, ovary, or testicle. In some examples, the cancer is a solid tumor cancer. For example, the cancer can include, but is not limited to: melanoma, sarcoma, melanoma, lymphoma, cancer of the breast, colon, bladder, prostate, lung, kidney, pancreas, liver, uterus, ovary, or testicle. In some examples, the cancer can be: B-lymphoma cells (RAJI) and breast cancer cells (TNBC: MDA-MB231-LN, HER2+: MDA-MB-361, SkBr3, BT-474).

The methods provided in the present disclosure are advantageous compared to existing technologies because the methods as disclosed herein identified prolyl hydroxylase inhibitory agents which can promote T cell stemness and suppresses T cell exhaustion. The methods described herein allow the application of the prolyl hydroxylase inhibitory agents as described herein during CAR-T manufacturing to generate modified CAR-T cells with higher proliferation potential and higher memory T cell percentage compared to conventional CAR-T cells.

Application of the methods disclosed herein therefore include, but are not limited to consultation and customization for optimization of cellular immunotherapy models in research and product development. Customized modified T cells (for example, STEM CAR-T cells or TCR-T cells) can be manufactured as commercial product to hospital, pharmaceutical company, or individuals.

The disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the disclosure embodied therein herein disclosed may be resorted to by those skilled in the art, and that such modifications and variations are considered to be within the scope of this disclosure.

It should further be appreciated that the exemplary embodiments are only examples, and are not intended to limit the scope, applicability, dimensions, or configuration of the disclosure in any way. Rather, the foregoing detailed description will provide those skilled in the art with a convenient road map for implementing an exemplary embodiment of the disclosure, it being understood that various changes may be made in the function and arrangement of elements and method of fabrication described in an exemplary embodiment without departing from the scope of the disclosure as set forth in the appended claims.

### EXPERIMENTAL SECTION

### Example 1: Conventional and STEM CAR-T Cell Preparation

### Day 0: T Cell Activation and Modification

1. Thaw peripheral blood mononuclear cells (PBMCs) or purified T cell.

2. Determine cell number needed.

3. For every 1×10⁶ cells, resuspend cells in 1 mL T cell activation medium (containing: 1 mL T cell culture medium (Stemcell Technology ImmunoCult™-XF T Cell Medium, catalogue number: 10981); 10 ng/ml human IL2; 10 μL of CD3/CD28 beads (Miltenyi Biotec T cell TransAct CD3/CD28 beads, catalogue number: 130-128-758). To obtain STEM CAR-T cells, 2 μM 1,4-DPCA-ester or any prolyl hydroxylase inhibitor can be added on day 0 of T cell activation to the T cell activation medium. Conventional CAR T cells are generated without addition of prolyl hydroxylase inhibitor or treated with vehicle for the prolyl hydroxylase inhibitor, which is DMSO.

4. Culture cells in tissue culture incubator at 37° C., 5% CO₂ for 5 days.

### Day 3: CAR Virus Transduction

Add 100 μL of concentrated viral supernatant to activated and modified T cells with polybrene at final concentration 6 μg/mL. A person skilled in the art would be able to adjust the amount of viral supernatant. Alternative methods of CAR virus transduction can be used.

### Day 5-Day 12: Conventional CAR-T and STEM CAR-T Cell Expansion

1. Collect all cells and perform a cell counting with a small fraction of the cells;

2. Centrifuge the remaining cells at 300 xg for 10 minutes to wash off the CAR virus and CD3/CD28 beads. Aspirate supernatant completely.

3. For every 0.25×10⁶ cells, resuspend cells in 1 mL a T cell expansion medium (contains: 1 mL T cell culture medium (Stemcell Technology ImmunoCult™-XF T Cell Medium, catalogue number: 10981); 10 ng/mL human IL7; 10 ng/mL human IL15 To the STEM CAR-T cells, culture 2 μM 1,4-DPCA-ester or any prolyl hydroxylase inhibitor can be added to T cell expansion medium may be added in this step, but the additional of 1,4-DPCA-ester or any prolyl hydroxylase inhibitor is optional in this step. Conventional CAR T cells are generated without addition of prolyl hydroxylase inhibitor or treated with vehicle for the prolyl hydroxylase inhibitor, which is DMSO.

4. T cell expansion can be continued at 0.25×10⁶ cells/mL of T cell expansion medium.

5. On day 7 and day 10, count and reseed cells at 0.25×10⁶ cell/mL with fresh T cell expansion medium. Optionally, 2 μM 1,4-DPCA-ester or any prolyl hydroxylase inhibitor can be added to T cell expansion medium for STEM CAR-T cell in this step.

6. Conventional CAR-T cells and STEM CAR-T cells will be ready for harvest and use from day 7 to day 12 whenever the cells reach a sufficient amount.

### Example 2: STEM-T Cell Modification/Reprogramming and Expansion

The STEM-T technology as disclosed herein is for modification of T cells in general, which could be applied to T cell based adoptive immunotherapies such as CAR-T or TCR-T therapies. An exemplary protocol for STEM-T cell modification/reprogramming and expansion is provided below.

### Day 0: T Cell Activation

1. Thaw PBMCs or purified T cell.

2. Determine cell number needed.

3. For every 1×10⁶ cells, resuspend cells in 1 mL T cell activation medium (containing: 1 mL T cell culture medium (Stemcell Technology ImmunoCult™-XF T Cell Medium, catalogue number: 10981); 2 μM 1,4-DPCA-ester or any prolyl hydroxylase inhibitor (added on day 0 of T cell activation); 10 ng/mL human IL2; 10 μL of CD3/CD28 beads (Miltenyi Biotec T cell TransAct CD3/CD28 beads, catalogue number: 130-128-758).

4. Culture cells in tissue culture incubator at 37° C., 5% CO₂ for 5 days.

### Day 5-Day 12: T Cell Expansion

1. Collect all cells and perform a cell counting with a small fraction of the cells.

2. Centrifuge the rest of the cells at 300 xg for 10 minutes to wash off the CD3/CD28 beads. Aspirate supernatant completely.

3. For every 0.25×10⁶ cells, resuspend cells in 1 mL a T cell expansion medium (contains: 1 mL T cell culture medium (Stemcell Technology ImmunoCult™-XF T Cell Medium, catalogue number: 10981); 10 ng/ml human IL7; 10 ng/ml human IL15; or optionally 2 μM 1,4-DPCA-ester or any prolyl hydroxylase inhibitor. The addition 1,4-DPCA-ester or any prolyl hydroxylase inhibitor is optional during T cell expansion step.

4. T cell expansion can be continued at 0.25×10⁶ cells/mL of T cell expansion medium.

5. On day 7 and day 10, count and reseed cells at 0.25×10⁶ cell/mL with fresh T cell expansion medium. Optionally, 2 μM 1,4-DPCA-ester or any prolyl hydroxylase inhibitor can be added to T cell expansion medium in this step.

6. STEM T cells will be ready for harvest and use from day 7 to day 12 whenever the cells reach a sufficient amount.

### Example 3: Co-Culture Experiment and Exhaustion Assay

Cancer cells were seeded in 24 well plate for 1 day to form monolayers. Culture media of cancer cell was then removed, and CAR T cells were subsequently added to the cancer cells 72 hours. Co-cultured CAR T cells were subsequently used for flow cytometry to check for PD1 and Tim3 expression for exhaustion cell population (that is PD1+Tim3+).

### Example 4: Co-Culture Cytotoxicity Assay

Coculture cytotoxicity assays were performed using a luciferase-based killing assay with CAR T cells incubated with cancer cells stably expressing luciferase at indicated effector to target (E:T) ratios. After 48 hours of co-culture, 1× luciferase substrate luciferinwas added to the cells and the chemiluminescent signals were detected by GlowMAX Explorer (Promega). The measurement was used to indicate cell viability against cancer cells without coculture. Percent cell cytotoxicity was calculated using the formula below:

$$\% cytotoxicity = 100 - ( \frac{Luciferase measurement of cocultured samples}{Luciferase measurement of noncocultured samples} ) \times 100 \%$$

### Example 5: CAR T-Cell and STEM CAR-T Cell Manufacturing

Anonymous human healthy donor peripheral blood mononuclear cells (PBMCs) were used for generation of conventional CAR-T cells and STEM CAR-T cells. To generate CAR-T cells, bulk PBMCs were activated on day 0 using 10 μL T Cell Transact containing CD3 and CD28 for each million PBMCs, cultured in ImmunoCult™-XF T Cell Expansion Medium (Stemcell Technologies; Cat. No. 10981) or any other suitable medium for culturing T cells, supplemented with 10 ng/mL recombinant human IL2 for 5 days. To generate STEM T-cells, prolyl hydroxylase inhibitor is added to the culture medium on day 0. The culture for conventional T cells is provided with an equivalent volume of DMSO on day 0. On day 3, cells were transduced with 100 μL CAR lentivirus in the presence of 6 μg/mL polybrene. CD3/CD28 agonist beads were removed on day 5 by washing the cells twice in 1×PBS. Cells were further expanded in ImmunoCult™-XF T Cell Expansion Medium containing 5 ng/mL recombinant human IL7 and 10 ng/ml recombinant IL15, maintaining a density of 0.25 million cells/mL from day 5 onwards. Subsequently, the conventional CAR T cells and STEM CAR T cells were sub-cultured under the same conditions every 3 days.

CAR-T cells are ready to be used on day 7.

To characterize the expansion rate and differentiation profiles of CAR-T cells, manual cell counts were conducted and recorded on day indicated.

### Example 6: Mouse Xenograft Model

NSG mice were injected with 2×10⁶ MDA-MB-231 cells, 5×10⁶ cells MDA-MB-361 cells or 2× 10⁶ DLD-1 cells suspended in 50 μL of phosphate buffer saline (PBS) to establish human breast or colon cancer models. ROR1, HER2 or EGFR1-targeting CAR T cells were injected into relevant mouse models at 2× 10⁶, 0.5×10⁶ and 2.5×10⁶ cells in 200 μL of PBS, respectively, through intravenous injection. The volume of the tumour on the mice were measured twice per week. Blood was collected from the mice through retro-orbital sinus for FACS analysis once per week. The data are shown in FIGS. 12-15 of the specification.

The examples set forth above are provided to give those of ordinary skill in the art a complete disclosure and description of how to make and use the embodiments of the compositions, systems and methods of the disclosure, and are not intended to limit the scope of what the inventors regard as their disclosure. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

Many modifications and variations of this application can be made without departing from its scope, as will be apparent to those skilled in the art. The specific embodiments and examples described herein are offered by way of example only, and the application is to be limited only by the terms of the appended claims, along with the full scope of equivalents to which the claims are entitled.

1. (canceled)

2. A method of preparing modified T cells, comprising modifying T cells during T cell activation by contacting the T cells with an inhibitory agent for prolyl hydroxylase, wherein the modifying optionally comprises increasing the expansion rate of T cells compared to unmodified T cells.

3. The method of claim 2, wherein the inhibitory agent for prolyl hydroxylase is a small molecule prolyl hydroxylase inhibitor.

4. The method of claim 2, wherein the modified T cells are used for adoptive T cell immunotherapy.

5. A method of producing Chimeric Antigen Receptor (CAR) T cells or T Cell Receptor (TCR)-T cells, comprising:
(a) modifying T cells during T cell activation by contacting the T cells with an inhibitory agent for prolyl hydroxylase;
(b) introducing a CAR transgene or a TCR transgene into the modified T cells of (a) to produce CAR-T cells or TCR-T cells; and
(c) harvesting the CAR-T cells or TCR-T cells after (b).

6. The method of claim 5, wherein steps (a) and (b) are carried out simultaneously.

7. The method of claim 2, wherein a time period for modifying the T cells is about 1 day to 5 days.

8.-9. (canceled)

10. The method of claim 5, wherein the harvested CAR-T cells or TCR-T cells are used for adoptive T cell immunotherapy.

11. The method of claim 2, wherein the inhibitory agent for prolyl hydroxylase is selected from the group consisting of 1,4-DPCA, 1,4-DPCA ethyl ester, Molidustat, Dencichine, Enarodustat, Roxadustat, IOX2, Daprodustat, and combinations thereof.

12.-14. (canceled)

15. A method of treating or alleviating a cancer in a subject, comprising administering a therapeutically effective amount of a CAR-T cell or a TCR-T cell produced by the method of claim 5 to a subject in need thereof.

16. The method of claim 15, wherein the method induces long-term anti-tumor effect in the subject.

17. The method of claim 15, wherein the method reduces tumor size in the subject.

18. The method of claim 15, wherein the method increases memory T cell and CAR-T cell stemness in vivo in the subject.

19. The method of claim 15, wherein the method increases tumor infiltration of T cells in the subject.

20. The method of claim 15, wherein the method increases the in vivo expansion rate of CAR-T cells or TCR-T cells in the subject.
